(19) United States
(12) Patent Application Publication
(10) Pub. No.: US 2008/0268460 A1
(43) Pub. Date: Oct. 30, 2008

(54) Assays to Identify Irreversibly Binding Inhibitors of Receptor Tyrosine Kinases

(75) Inventors: Frank Loganzo, New City, NY (US); Lee M. Greenberger, Montclair, NJ (US); Xingshi Tan, Congers, NY (US); Allan Wissner, Ardsley, NY (US)

Correspondence Address: Wyeth c/o Darby & Darby, P.C., P.O. BOX 770, Church Street Station, NEW YORK, NY, 10008-0770, US

(73) Assignee: Wyeth, Madison, NJ (US)

(21) Appl. No.: 11/569,288

(22) PCT Filed: May 11, 2005
(86) PCT No.: PCT/US05/16951

§ 371 Date: Feb. 8, 2007

Related U.S. Application Data
(60) Provisional application No. 60/573,240, filed on May 20, 2004.

(51) Int. Cl. G01N 33/53

(52) U.S. Cl. 435/7.1

(57) ABSTRACT

The present invention relates to a method of identifying an inhibitor of a receptor tyrosine kinase that irreversibly binds to the kinase. Specifically, the method comprises using a variety of assays, either alone or in combination, to identify compounds that irreversibly bind to tyrosine kinases. More specifically, there are four assays, which are novel variations of a basic enzyme assay and identify irreversible binding inhibitors.

This application claims priority from U.S. Provisional Application Ser. No. 60/573,240, filed May 20, 2004, the disclosure of which is incorporated herein by reference in its entirety.

### 1. FIELD OF THE INVENTION

The present invention relates to assays capable of identifying inhibitors of receptor tyrosine kinases that irreversibly bind to the tyrosine kinases, especially inhibitors of vascular endothelial growth factor receptor-2 (VEGR-2), also known as KDR.

### 2. BACKGROUND OF THE INVENTION

While the use of chemotherapy in treating cancer patients with later stage disease has extended survival, in many instances, it is at the cost of a poor quality of life. As a result, novel approaches of treating cancer by identifying selective targets has evolved. It is hoped that by using selective targets, the cancer can be cured, or at the very least, the progression of the cancer slowed or stopped, allowing the patient to live with his or her disease, while enjoying an acceptable quality of life.

Angiogenesis or the process of new blood vessel growth is required for the growth of primary tumors, as well as the metastasis of tumors. Angiogenesis of tumors allows them access to blood-derived oxygen and nutrients, and also provides them adequate perfusion. Hence inhibiting angiogenesis is an important therapeutic strategy in treating cancer. Inhibition of angiogenesis is also therapeutically useful in treating other chronic diseases such as rheumatoid arthritis, psoriasis, diabetic retinopathy and age-related macular degeneration.

Tumor cells produce a number of angiogenic molecules, including vascular endothelial growth factor (VEGF). Data supports the role of VEGF (ligand) and KDR (receptor) in tumor angiogenesis and metastasis. VEGF is secreted by many cancer cell lines in vitro and by their tumors in vivo. In patients, the expression of VEGF in solid tumors and KDR in leukemia negatively correlates with survival.

VEGF is a homodimeric disulfide-linked member of the PDGF family, an endothelial cell-specific mitogen known to cause a profound increase in the vascular endothelial permeability in the affected tissues. VEGF is also a senescence-preventing survival factor for endothelial cells. Almost all nucleated tissues in the body possess the capability to express VEGF in response to various stimuli including hypoxia, glucose deprivation, advanced glycation products and inflammatory cytokines.

Growth-promoting angiogenic effects of VEGF are mediated predominantly via its signaling receptor called kinase insert domain containing receptor or KDR. This receptor is also referred to as Flk-1 or VEGFR-2. KDR is a receptor protein tyrosine kinase with an extracellular VEGF-binding domain consisting of seven immunoglobulin-like domains and a cytoplasmic domain containing the catalytic tyrosine kinase domain split by a kinase-insert region. The expression of KDR is low on most endothelial cells; however, activation with angiogenic agents results in a significant upregulation of KDR on endothelial cells. Most angiogenized blood vessels express high levels of KDR. Binding to VEGF causes dimerization of KDR resulting in its autophosphorylation and initiation of signaling cascade. Therefore, tyrosine kinase activities of KDR are essential for mediation of the functional effects of VEGF.

The sequence of KDR DNA and protein are known in the art and described at least in the following references: Yilmaz, A. et al. “p38 MAPK inhibition is critically involved in VEGFR-2-mediated endothelial cell survival” Biochem. Biophys. Res. Commun. 306(3):730-736 (2003); Zeng, H. et al. “Heterotrimeric G alpha q/G alpha 11 proteins function upstream of vascular endothelial growth factor (VEGF) receptor-2 (KDR) phosphorylation in vascular permeability factor/VEGF signaling” J. Biol. Chem. 278(23):20738-20745 (2003); Yang, S. et al. “Vascular endothelial growth factor-induced genes in human umbilical vein endothelial cells: relative roles of KDR and Flt-1 receptors” Arterioscler. Thromb. Vasc. Biol. 22(11):1797-1803 (2002); U.S. Pat. No. 5,861,301, issued Jun. 19, 1999 to Terman et al., entitled “Recombinant Kinase Insert Domain Containing Receptor and Gene Encoding the Same”; U.S. Pat. No. 5,766,860, issued Jun. 16, 1998 to Terman et al., entitled “Screening Method Using a Recombinant Kinase Insert Domain Containing Receptor and Gene Encoding the Same”; and Terman, B. I. et al. “Identification of the KDR tyrosine kinase as a receptor for vascular endothelial cell growth factor” Biochem. Biophys. Res. Commun. 187(3):1579-1586 (1992). The full mRNA and protein sequence of KDR can be found in GenBank, accession numbers NM_{—}002253 and NP_{—}002244.1, respectively. Furthermore, a computer model of the crystal structure of KDR has also been reported. McTigue et al. “Crystal structure of kinase domain of human vascular endothelial growth factor receptor 2: a key enzyme in angiogenesis” Structure 7:319-330 (1999).

Compounds that inhibit the tyrosine kinase activity of KDR will also function as anti-angiogenic agents and are useful for the treatment of cancer and other diseases characterized by excessive, abnormal or inappropriate angiogenesis. Neutralizing antibodies to VEGF and KDR inhibit primary tumor growth, as well as metastases, in vivo. When these neutralizing antibodies are used in combination with standard cytotoxics, such as paclitaxel, efficacy of the cytotoxics is improved. Antisense RNA, ribozymes and DNAzyme technology that specifically diminish VEGR or KDR expression have been demonstrated to be effective in both cellular and animal models.

Some small molecule inhibitors of KDR kinase are also in development. Unlike RNA and antibody strategies, most of the small molecule inhibitors are non-selective and inhibit other related kinases, which may be of benefit since some of these kinases also may be involved in angiogenesis. These agents appear to be most effective when administered orally on a daily basis.

There are several benefits to the use of anti-angiogenic therapy. Genetically unstable cancer cells often develop resistance to standard therapy. By targeting untransformed endothelial cells, resistance is less likely to develop. Additionally, slow growing tumors that are resistant to standard cytotoxic cancer therapy may be responsive to a continuous low to moderate dose of anti-angiogenic drugs. Moreover, since the therapeutic target is not the tumor cell itself, the anti-angiogenic drug therapy is effective against tumors from different tissue origins. The growth of solid tumors, such as lung, colorectal, breast and prostate, have been inhibited by targeting KDR in animal models as well as patients.

However, despite these benefits, the clinical results of the inhibitor therapy has been mixed. Phase I safety trials of small molecules and antibody monotherapy has shown minimal adverse side effects. However, combination trials with established cytotoxic therapy have resulted in more adverse events, such as vascular effects. In phase II and III clinical trials of solid tumors, some partial regressions have been observed. Some complete regressions, increased time to progression and increased survival time have been reported with the anti-VEGF antibody, alone or in combination therapy.

For recent reviews on this subject, see F. J. Giles “The Emerging Role of Angiogenesis Inhibitor in Hematologic Malignancies” Oncology, Supplement 16:23-29 (2002); S. J. Boyer “Small Molecule Inhibitors of KDR (VEGFR-2) Kinase: An Overview of Structure Activity Relationships” Curr. Top. Med. Chem., 2:973-1000 (2002); J. Folkman “Role of Angiogensis in Tumor Growth and Metastasis” Seminars in Oncology 29:15-18 (2002); and R. K. Jain “Tumor Angiogenesis and Accessibility: Role of Vascular Endothelial Growth Factor” Seminars in Oncology 29:3-9 (2002).

It is unknown why there is limited success with these agents. However, an alternative method of targeting KDR is to use irreversible binding inhibitors. The KDR inhibitors known to date are believed to reversibly bind to the target receptor, but compounds that irreversibly bind to certain other target receptors have been shown to be superior tumor suppressors. For example, Frey et al. (Proc. Natl. Acad. Sci. U.S.A. 95:12022-12027 (1998)) have reported that small molecules purported to irreversibly inhibit epidermal growth factor receptor (EGFR) also bind irreversibly to the receptor and alkylate a cysteine residue in the ATP binding pocket of the molecule. These compounds are said to be more potent suppressors of tumor growth in animal models. Others have reported that irreversible EGFR kinase inhibitors effectively suppress growth in human tumor cell models (Discafani et al., Biochem. Biopharmacol. 57:917-925 (1999)). Hence, the identification of compounds that irreversibly bind KDR offers the ability to identify new therapeutic compounds which are likely to be superior tumor suppressors compared to the reversible KDR inhibitors that are currently available.

A variety of assay platforms are already available that can identify inhibitors of a tyrosine kinase protein. For example, enzyme-linked immunosorbent assay (ELISA) platforms are known in which a horseradish conjugated anti-phosphotyrosine antibody is used to detect phosphorylation of a biotin-conjugated peptide substrate immobilized on a solid phase plate. A similar assay platform is also marketed by PerkinElmer Lifesciences (Wellesley, Mass.) under the tradename DELFIA® (for dissociation enhanced lanthanide fluorescent immunoassay). The DELFIA® platform is distinguishable from ELISA in that it uses a europium-labeled, rather than an enzyme-conjugated, anti-phosphotyrosine antibody. See, for example, Loganzo & Hardy, American Biotechnology 16:26-28 (1998). Other assay platforms for tyrosine kinase activity are described, e.g., in U.S. Pat. No. 6,066,462 by Goueli, issued May 23, 2000. These assays perform a kinase reaction in the presence of ³²P-labeled ATP, and then use liquid scintillation spectrophotometry to measure ³²P incorporation in an immobilized peptide substrate.

However, none of these assay platforms specifically identifies irreversible inhibitors of a tyrosine kinase. In particular, the assays cannot distinguish between compounds that inhibit tyrosine kinase activity by either irreversible or reversible binding.

There have been reports of other assay types, specifically those using cell extracts and Western blotting, to screen for irreversible kinase inhibitors, namely those to EGFR. See, for example, International Patent Application No. WO 97/38983 and Smaill et al., Journal of Medicinal Chemistry 43:1380-97 (2000). However, this type of assay would be more labor intensive and cumbersome than the ELISA or DELFIA® format.

Hence, there is a need in the art for effective and efficient screening assays and platforms that can identify compounds that irreversibly inhibit a tyrosine kinase, e.g. by binding irreversibly to that enzyme. More specifically, there is a need for effective and efficient screening assays and platforms that can identify compounds that inhibit tyrosine kinase receptor proteins such as KDR.

The citation and/or discussion of a reference in this section and throughout the specification is provided merely to clarify the description of the present invention and is not an admission that any such reference is “prior art” to the invention described herein.

### 3. SUMMARY OF THE INVENTION

The present invention overcomes the above and other problems in the art by providing assays that identify compounds that are potent inhibitors of tumor cell growth and proliferation. In particular, the invention provides assays that identify compounds which both inhibit a tyrosine kinase enzyme and irreversibly bind to that target. In a preferred embodiment, the invention provides assays that identify compounds which irreversibly bind to and inhibit a VEGF receptor, such as KDR.

One embodiment of the invention provides for an assay for identifying a compound which binds irreversibly to a tyrosine kinase enzyme, by (a) incubating a mixture comprising the tyrosine kinase enzyme and a test compound in a substrate-coated plate well under conditions wherein, in the absence of the test compound, phosphorylation of the substrate by the tyrosine kinase enzyme would normally occur; (b) adding a wash solution to the mixture of step a) to wash out any test compound not bound to the tyrosine kinase enzyme; (c) adding ATP to the mixture of step a); (d) incubating the plate wells with an antibody to the phosphorylated substrate, wherein the antibody is coupled to a label; (e) detecting the amount of phosphorylated substrate; and (f) determining the level of phosphorylated substrate in the presence of the test compound after step b) relative to the level of phosphorylated substrate in the presence of the test compound in a sample performed without step b), wherein a difference of about three-fold or less indicates that the test compound binds irreversibly to the tyrosine kinase enzyme.

In a more preferred embodiment, the difference between the level of phosphorylated substrate in the presence of the test compound after step b) and the level of phosphorylated substrate in the presence of the test compound in a sample performed without step b) is two-fold or less.

A further embodiment of the present invention is another assay for identifying a compound which binds irreversibly to a tyrosine kinase enzyme by looking at the compound's ability to compete with ATP. This assay includes the steps of (a) incubating a mixture comprising the tyrosine kinase enzyme and a test compound in a substrate-coated plate well under conditions wherein, in the absence of the test compound, phosphorylation of the substrate by the tyrosine kinase enzyme would normally occur; (b) adding ATP to the mixture of step a), in at least two increasing varying concentrations; (c) incubating the plate wells with an antibody to the phosphorylated substrate, wherein the antibody is coupled to a label; (d) detecting the amount of phosphorylated substrate; and (e) determining the level of phosphorylated substrate in the presence of the test compound and the varying increasing concentrations of ATP, wherein a change of about three-fold or less in the level of phosphorylation of the substrate in the varying increasing concentrations of ATP indicates that the test compound does not compete with ATP and binds irreversibly to the tyrosine kinase enzyme.

A preferred embodiment of this assay includes using more than two varying increasing concentrations of ATP, preferably three, and most preferably four.

A further embodiment of the invention is a third assay for the identification of a compound which binds irreversibly to a tyrosine kinase enzyme, by (a) incubating a mixture comprising a tyrosine kinase enzyme and a test compound and subjecting the mixture to dialysis; (b) placing the dialyzed mixture in a substrate-coated plate well under conditions wherein, in the absence of the test compound, phosphorylation of the substrate by the tyrosine kinase enzyme would normally occur; (c) adding ATP to the reaction mixture of step a); (d) incubating the plate wells with an antibody to the phosphorylated substrate, wherein the antibody is coupled to a label; (e) detecting the amount of phosphorylated substrate; and (f) determining the level of phosphorylated substrate in the presence of the test compound in the mixture subject to dialysis relative to the level of phosphorylated substrate in the presence of the test compound not subject to dialysis, wherein a difference of about three-fold or less indicates that the test compound binds irreversibly to the tyrosine kinase enzyme.

In another embodiment of the present invention, another assay for the identification of an irreversibly binding inhibitor of a tyrosine kinase enzyme is provided that includes performing the steps of (a) incubating a mixture comprising the tyrosine kinase enzyme that comprises at least one altered amino acid and a test compound in a substrate-coated plate well under conditions wherein, in the absence of the test compound, phosphorylation of the substrate by the tyrosine kinase enzyme would normally occur; (b) adding ATP to the reaction mixture of step a); (c) incubating the plate wells with an antibody to the phosphorylated substrate, wherein the antibody is coupled to a label; (d) detecting the amount of phosphorylated substrate; and (e) determining the level of phosphorylated substrate in the presence of the test compound and the tyrosine kinase enzyme comprising at least one altered amino acid relative to the level of phosphorylated substrate in the presence of the test compound and a tyrosine kinase enzyme with no altered amino acids, wherein a decrease in the level of phosphorylation of the substrate indicates that the test compound binds to the amino acid in the tyrosine kinase enzyme that has been altered and binds irreversibly to the unaltered tyrosine kinase enzyme.

These assays can be performed individually to determine or confirm if a test compound is an irreversible binding inhibitor of tyrosine kinase. A preferred embodiment is that at least two assays are performed to identify irreversible binding inhibitor compounds and more preferred that three are performed. In the most preferred embodiment, it is contemplated that the first three assays are performed and then the fourth assay is performed to confirm irreversible binding involves covalent binding to a particular amino acid residue.

While these assays can be used to identify irreversibly binding inhibitors of many receptor tyrosine kinases, the preferred kinase is KDR.

The assays described herein may be used in a high-throughput primary screen for irreversible binding inhibitors of tyrosine kinases, or it may be used as a secondary functional screen for candidate compounds identified by a different primary screen, e.g., a screen that identifies compounds that inhibit receptor tyrosine kinases, whose binding capacity is not known, or as an assay to confirm irreversible binding of an inhibitor compound to a receptor tyrosine kinase.

### 4. BRIEF DESCRIPTION OF THE DRAWINGS

FIG. 1 shows the X-ray structure of the catalytic domain of KDR, including the cysteine 1045 and lysine 868 amino acid residues, which can be altered to obtain mutated forms of the KDR enzyme.

FIG. 2 shows the results of an enzyme assay using the KDR enzyme and test compound, 2-[4-(1H-imidazol-1-yl)phenoxyl]-5-{6-methoxy-7-(2-methoxyethoxy)quinazolin-4-yl]amino}benzo-1,4-quinone, and varying concentrations of ATP. The X axis depicts the concentration of test compound and the Y axis depicts the percent inhibition. The four different curves represent the four different concentrations of ATP used in the assay.

### 5. DETAILED DESCRIPTION

There are no reported small molecule inhibitors of KDR that irreversibly bind to the kinase. Using computer modeling based upon the published crystal structure of I<DR (McTigue et al. “Crystal structure of kinase domain of human vascular endothelial growth factor receptor 2: a key enzyme in angiogenesis” Structure 7:319-330 (1999)), we developed irreversible binding inhibitor compounds of KDR. These compounds are described in patent application Ser. No. 60/573,251, entitled “QUINONE SUBSTITUTED QUINAZOLINE AND QUINOLINE KINASE INHIBITORS”, by inventors Allan Wissner, Bernard Dean Johnson, Heidi Leigh Fraser, Russell George Dushin, Charles Ingalls, Ramaswamy Nilakantan, Middleton Brawner Floyd Jr. and Thomas Naittoli, filed concurrently herewith.

There are many advantages to an irreversible KDR inhibitor. For one, these inhibitors would not compete with ATP. A tyrosine kinase such as KDR catalyzes the transfer of a phosphate group from a molecule of ATP to a tyrosine residue located on a protein substrate. The inhibitors of KDR so far known in the art are reversible and usually competitive with either ATP or the protein substrate of the kinase, or both simultaneously. Since the concentration of ATP in a cell is normally very high (millimolar), compounds that are competitive with ATP may show diminished efficacy and duration of action since it would be difficult for such compounds to reach the concentrations within the cell that are necessary to displace the ATP from its binding site for the extended time needed to inhibit tumor growth effectively. Compounds which inhibit tyrosine kinases and bind in an irreversible manner would be non-competitive with ATP or protein substrate.

Secondly, since prolonged suppression of the kinase is most likely necessary for maximum tumor suppression, an irreversibly bound inhibitor provides an advantage by permanently eliminating the existing kinase activity, which should return only when a new receptor is synthesized.

Lower plasma levels of the inhibitor is also an advantage. The irreversible binding inhibitors require that plasma concentrations be attained only long enough to expose the inhibitor to the target. After the irreversible inhibitor binds, no more inhibitor is needed in the plasma in order to maintain inhibition. Thus, there is less likelihood of toxicity, which results from high or prolonged plasma levels.

Lastly, there may be possible cross-reactivity of the irreversible binding inhibitors with other kinases involved in angiogenesis that have homologous amino acids in their active site, e.g., platelet-derived growth factor receptor (PDGFR) and vascular endothelial growth factor receptor 1 (VEGFR-1).

The present invention is directed to a number of assays for the identification of compounds that irreversibly bind to receptor tyrosine kinases, in particular, VEGFR-2 or KDR. The four assays are: (1) compound wash-out in an enzyme assay; (2) ATP competition studies in an enzyme assay; (3) dialysis of the enzyme and the test compound and analysis using an enzyme assay; and (4) the use of a mutated receptor tyrosine kinase in an enzyme assay, or in any of the three preceding three assays.

Any one of these four listed assays can show that the test compound likely irreversibly binds to the tyrosine kinase. However, it is preferred that at least two are performed, more preferably three, and most preferably all four. A positive result on all four assays means there is a high likelihood that the inhibitor compound binds irreversibly to the kinase.

### DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to the preferred embodiments.

“Irreversible” or “irreversibly” as the terms are used herein mean an inhibitor of receptor tyrosine kinase activity that is permanently bound or associated with the receptor tyrosine kinase.

“Identify” as the term is used herein means either screening for a compound that may irreversibly bind to a tyrosine kinase inhibitor, i.e., the assay is performed to determine whether the inhibitor irreversibly binds to the tyrosine kinase enzyme, or an assay performed to further characterize a known irreversible inhibitor or elucidate a mechanism of action.

“Test compound” is a molecule that can be tested for its ability to irreversibly bind to a tyrosine kinase enzyme or further characterized as to its irreversible binding to a tyrosine kinase enzyme.

“Under conditions wherein, in the absence of the test compound, phosphorylation of the substrate by the tyrosine kinase enzyme would normally occur” will be understood by a person of skill in the art as the conditions, such as time, temperature and pH, that are necessary for normal phosphorylation of the substrate by the tyrosine kinase enzyme to take place.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

An “enzyme” is considered a protein and refers to polypeptides that contain the amino acid residues encoded by a gene or by a nucleic acid molecule (e.g., an mRNA or a cDNA) transcribed from that gene either directly or indirectly. Optionally, a protein may lack certain amino acid residues that are encoded by a gene or by an mRNA. For example, a gene or mRNA molecule may encode a sequence of amino acid residues on the N-terminus of a protein (i.e., a signal sequence) that is cleaved from, and therefore may not be part of, the final protein. A protein or polypeptide, including an enzyme, may be a “native” or “wild-type”, meaning that it occurs in nature; or it may be a “mutant”, “variant”, “modified” or “altered” meaning that it has been made, derived, or is in some way different or changed from a native protein or from another mutant.

The preferred tyrosine kinase enzymes for which the assays identify irreversible inhibitors are described as follows. The protein sequence for VEGFR-2 or KDR is found in GenBank, accession number NM_{—}002253 (mRNA) and NP_{—}002244.1 (protein) and has been described, at least, in Yilmaz, A. et al. “p38 MAPK inhibition is critically involved in VEGFR-2-mediated endothelial cell survival” Biochem. Biophys. Res. Commun. 306(3):730-736 (2003); Zeng, H. et al. “Heterotrimeric G alpha q/G alpha 11 proteins function upstream of vascular endothelial growth factor (VEGF) receptor-2 (KDR) phosphorylation in vascular permeability factor/VEGF signaling” J. Biol. Chem. 278(23):20738-20745 (2003); Yang, S. et al. “Vascular endothelial growth factor-induced genes in human umbilical vein endothelial cells: relative roles of KDR and Flt-1 receptors”, Arterioscler. Thromb. Vasc. Biol. 22(11):1797-1803 (2002); U.S. Pat. No. 5,861,301, issued Jun. 19, 1999 to Terman et al., entitled “Recombinant Kinase Insert Domain Containing Receptor and Gene Encoding the Same”; U.S. Pat. No. 5,766,860, issued Jun. 16, 1998 to Terman et al., entitled “Screening Method Using a Recombinant Kinase Insert Domain Containing Receptor and Gene Encoding the Same”; and Terman, B. I. et al. “Identification of the KDR tyrosine kinase as a receptor for vascular endothelial cell growth factor” Biochem. Biophys. Res. Commun. 187(3):1579-1586 (1992). The protein sequence of VEGFR-2 or KDR is reproduced as SEQ. ID. NO. 1.

The sequence of vascular endothelial growth factor receptor-1 or VEGFR-1 is found in GenBank, accession number NM_{—}002019 (mRNA) and NP_{—}002010 (protein) and has been described, at least, in Wang et al. “Homeostatic modulation of cell surface KDR and Flt1 expression and expression of the vascular endothelial cell growth factor (VEGF) receptor mRNAs by VEGF” J. Biol. Chem. 275(21):15905-15911 (2000); and Herley, M. T. et al. “Characterization of the VEGF binding site on the Flt-1 receptor” Biochem. Biophys. Res. Commun. 262(3):731-738 (1999). The protein sequence of VEGR-1 is reproduced as SEQ. ID. NO. 2.

The sequence of vascular endothelial growth factor receptor-3 (VEGFR-3) is found in GenBank, accession number NM_{—}182925 (mRNA) and NP_{—}891555 (protein) and has been described, at least, in Hamrah, P. et al. “Novel expression of vascular endothelial growth factor receptor (VEGFR)-3 and VEGF-C on corneal dendritic cells” Am. J. Pathol. 163(1):57-68 (2003); and Witte, D. et al. “Expression of the vascular endothelial growth factor receptor-3 (VEGFR-3) and its ligand VEGF-C in human colorectal adenocarcinoma” Anticancer Res. 22(3):463-1466 (2002). The protein sequence of VEGFR-3 is reproduced as SEQ. ID. NO. 3.

The sequence of platelet derived growth factor receptor (PDGFR) is found in GenBank, accession number NM_{—}002609 (mRNA) and NP_{—}002600 (protein) and has been described, at least, in Matsui, T. et al. “Isolation of a novel receptor cDNA establishes the existence of two PDGF receptor genes” Science 243(4892):800-804 (1989); Claesson-Welsh, L. et al. “cDNA cloning and expression of a human platelet-derived growth factor (PDGF) receptor specific for B-chain-containing PDGF molecules” Mol. Cell. Biol. 8(8):3476-3486 (1988); and Gronwald, R. G. et al. “Cloning and expression of a cDNA coding for the human platelet-derived growth factor receptor: evidence for more than one receptor class” Proc. Natl. Acad. Sci. U.S.A. 85(10):3435-3439 (1988). The protein sequence of PDGR has been reproduced as SEQ. ID. NO. 4.

The sequence of fibroblast growth factor receptor (FGFR) is found in GenBank, accession number NM_{—}015850 (mRNA) and NP_{—}056934 (protein) and has been described, at least, in Groth, C. and Lardelli, M. “The structure and function of vertebrate fibroblast growth factor receptor 1” Int. J. Dev. Biol. 46(4):393-400 (2002); and Johnson, D. E. and Williams, L. T. “Structural and functional diversity in the FGF receptor multigene family” Adv. Cancer Res. 60:1-41 (1993). The protein sequence of FGFR is reproduced as SEQ. ID. NO. 5.

The sequence of epidermal growth factor receptor (EGFR) is found in GenBank, accession number NM_{—}005228 (mRNA) and NP_{—}005219 (protein) and has been described, at least, in Pennock, S, and Wang, Z. “Stimulation of cell proliferation by endosomal epidermal growth factor receptor as revealed through two distinct phases of signaling” Mol. Cell. Biol. 23(16):5803-5815 (2003); and Wang, X. et al. “Epidermal growth factor receptor is a cellular receptor for human cytomegalovirus” Nature 424(6947):456-461 (2003). The protein sequence of EGFR is reproduced as SEQ. ID. NO. 6.

It will be understood by those in the art that the assays and methods of the present invention can be practiced using proteins that are “homologous” to or are “homologs” of the tyrosine kinase enzymes. The terms “homologous” and “homologs”, in all their grammatical forms and spelling variations, refers to the relationship between two proteins that possess a “common evolutionary origin”, including proteins from superfamilies (e.g., the immunoglobulin superfamily) in the same species of organism, as well as homologous proteins from different species of organism (for example, myosin light chain polypeptide, etc.; see, Reeck et al., Cell 1987, 50:667). Such proteins (and their encoding nucleic acids) have sequence homology, as reflected by their sequence similarity, whether in terms of percent identity or by the presence of specific residues or motifs and conserved positions.

It will also be understood that orthologs of the enzymes can also be used in the present invention. As used herein, the term “orthologs” refers to genes in different species that apparently evolved from a common ancestral gene by speciation. Normally, orthologs retain the same function through the course of evolution. Identification of orthologs can provide reliable prediction of gene function in newly sequenced genomes. Sequence comparison algorithms that can be used to identify orthologs include without limitation BLAST, FASTA, DNA Strider, and the GCG pileup program. Orthologs often have high sequence similarity.

The Basic Enzyme Assay

All of the assays used to test for the irreversible binding of an inhibitor compound are based on the use of an immunoassay utilizing a label for detection of a reaction, particularly kinase phosphorylation. Thus, any enzyme assay that detects kinase phosphorylation can be used. Such assays include an enzyme linked immunoassay or ELISA and a dissociation enhanced lanthanide fluorescent immunoassay or DELFIA®. Labels that can be used include fluorescence, P³² and peroxidase. Many of these types of assays are sold as kits, such as the DELFIA®, sold by PerkinElmer and an ELISA, sold by Roche Diagnostics. Other kinase assay kits are sold by Cell Signaling, Inc. and CalBiochem/Oncogene Science. Components that can be used in the assay are sold by many companies known to those of skill in the art. This assay will be referred to herein as “the basic enzyme assay.”

In performing the assay, the tyrosine kinase enzyme is incubated with a test compound in a substrate-coated plate well. The term “substrate” as used herein means the substance upon which the enzyme acts. The preferred substrate is poly(Glu₄-Tyr) polypeptide. However, other substrates known in the art may be used, such as poly(Glu₄-Ala-Tyr), as well as peptides derived from the autophosphorylation site of kinases or the phosphorylation site of known substrates.

Examples of the tyrosine kinase enzyme are vascular endothelial growth factor receptor-1 (VEGFR-1) (SEQ. ID. NO. 2), vascular endothelial growth factor receptor-2 (VEGFR-2 or KDR) (SEQ. ID. NO. 1), vascular endothelial growth factor receptor-3 (VEGFR-3) (SEQ. ID. NO. 3), platelet derived growth factor receptor (PDGFR) (SEQ. ID. NO. 4), fibroblast growth factor receptor (FGFR) (SEQ. ID. NO. 5) and endothelial growth factor receptor (EGFR) (SEQ. ID. NO. 6) and their homologs and orthologs. However, other tyrosine kinase enzymes known in the art of which inhibitor compounds that irreversibly bind are desired can be used in the assays. The preferred tyrosine kinase enzyme to be used is KDR (SEQ. ID. NO. 1).

The tyrosine kinase enzyme can be prepared by recombinant methods known in the art. Such techniques are explained fully in the literature. See, e.g., Sambrook, Fritsch & Maniatis, Molecular Cloning: A Laboratory Manual, Second Edition. Cold Spring Harbor, N.Y.: Cold Spring Harbor Laboratory Press, 1989; DNA Cloning: A Practical Approach, Volumes I and II (D. N. Glover, ed. 1985); Oligonucleotide Synthesis (M. J. Gait, ed. 1984); Nucleic Acid Hybridization (B. D. Hames & S. J. Higgins, eds. 1985); Transcription And Translation (B. D. Hames & S. J. Higgins, eds. 1984); Animal Cell Culture (R. I. Freshney, ed. 1986); Immobilized Cells And Enzymes (IRL Press, 1986); B. Perbal, A Practical Guide To Molecular Cloning (Ausubel, F. M. et al., eds. 1984); Current Protocols in Molecular Biology (John Wiley & Sons, Inc., 1994).

For example, the KDR protein was prepared by isolating total mRNA from human umbilical vein endothelial cells and generating cDNA using real time polymerase chain reaction. The cDNA was cloned into a vector and transfected into human embryonic kidney cells. The vector further contained a tag sequence, in this case the FLAG sequence, to be used in the subsequent protein purification. The cells were grown up and the protein isolated from the cell lysate using anti-FLAG M2 affinity resin. The I<DR protein was also expressed in Sf9 insect cells using an N-terminal GST-His protein tag.

Other tags can be used to facilitate the protein purification. These tags are known in the art and include, among others, a-tubulin, B-tag, E-tag, c-myc, FLAG epitope, HA, HSV, PK-tag, Protein C, T7, VSV-G, GST and His. The use of these tags is optional. Furthermore, the tags can be used alone or in combination.

The tyrosine kinase enzyme may also be obtained by standard protein purification methods known in the art from cells that express these kinases, including, but not limited to, endothelial cells and tumor cells. The proteins can be purified by various methods including, without limitation, affinity chromatography, preparative disc-gel electrophoresis, isoelectric focusing, HPLC, reversed-phase HPLC, gel filtration, ion exchange and partition chromatography, precipitation and salting-out chromatography, extraction, and countercurrent distribution.

The next step of the basic enzyme assay is to add ATP to initiate the reaction where the tyrosine kinase phosphorylates the substrate. ATP is added so that the final concentration of the ATP in the reaction is from about 1 nM to 10 mM, with the preferred concentration being from about 0.1 uM to 100 uM, and the most preferred concentration being 10 uM.

After a washing step, an antibody coupled to a label is added to the wells. The antibody should recognize the phosphorylated substrate. An example of such an antibody is an anti-phosphotyrosine antibody designated PT66 and available from PerkinElmer. The antibody needs to be labeled for detection. One such label is a fluorescent label. The term “fluorescent label” as used herein would mean a substance or a portion of a substance that is capable of exhibiting fluorescence in a detectable range. Examples of such a label are europium, terbium, dysprosium and samarium.

Other suitable labels for use in the basic enzyme assay include enzymes, fluorophores, chromophores, radioisotopes, dyes, colloidal gold, colloidal carbon, latex particles, and chemiluminescent agents.

Lastly, the amount of phosphorylated substrate is detected. This is done by measuring the labeled antibody by any suitable method known in the art. For example a fluorescent signal can be measured using a fluorometer.

The level of the phosphorylated substrate in the presence of the test compound is compared to the level of the phosphorylation in the absence of the test compound. A decrease in the level of the phosphorylation indicates that the test compound is a compound that inhibits tyrosine kinase activity. The inhibition is generally represented by percent inhibition or IC₅₀.

To test the stability of the compounds in a reducing environment such as a cell, the basic enzyme assay should be performed in reducing conditions. Reducing agents, such as DTT, beta-mercaptoethanol, L-cysteine and glutathione, can be added to the assay during the incubation step of the test compound and the kinase. The assay is then performed as described above. If there is no significant difference between the percent inhibition of the sample where the reducing agent is used and one where it is not, then the test compound is considered to be stable in a reducing environment, e.g., a cell. The preferred reducing compound to be used in such an assay is glutathione at a concentration of 100 uM.

In order to determine whether a compound found to inhibits and binds irreversibly to the tyrosine kinase, the basic enzyme assay is modified, which results in the four assay protocols set forth below.

The Wash-Out Enzyme Assay

The first assay uses the basic enzyme assay but includes an additional washing step after the pre-incubation of the tyrosine kinase enzyme and the test compound, but prior to the addition of the ATP to initiate the reaction. The principle being that if there is still inhibition of kinase activity by the test compound after washing of unbound compound, the binding of the inhibitor to the test compound likely is irreversible. The washing step can be done with any conventional washing solution used in the art, but is preferably a buffer and the preferred buffer is HEPES at a pH of 7.4. Moreover, it can be performed once or multiple times. The washed-out sample of test compound is tested against an unwashed sample, i.e., a sample tested using the basic enzyme assay. Generally a difference of IC₅₀ of about three-fold or less, and preferably two-fold or less, between the washed-out and unwashed test identifies a test compound as binding irreversibly.

### The ATP Competition Enzyme Assay

It is also predicted that inhibitors of receptor tyrosine kinases that bind tightly and irreversibly would not be affected by ATP, even at high concentrations. To test this parameter, ATP is added in the basic enzyme assay to achieve varying increasing final concentrations and the percent inhibition is determined for each concentration of ATP. At least two different samples with different concentration levels of ATP need to be performed but more than two is preferable. The range of final concentrations of ATP can be from about 1 nM to 10 mM. A preferred embodiment of the assay uses four final concentrations of about 1, 10, 100 and 1000 uM of ATP.

Generally differences of the IC₅₀ of the test compound of three-fold or less for the increasing concentrations of ATP is an indication that the compound does not compete with ATP and is another indication that the compound likely binds irreversibly to the tyrosine kinase.

Some compounds in which increasing concentrations of ATP do not affect inhibition do not actually compete with ATP. In other words, the inhibitor compound may bind to the peptide-binding site, rather than the ATP-binding site, of the enzyme. Most compounds that inhibit tyrosine kinase receptor enzymes reversibly bind to the enzyme and most are competitive with ATP. Thus, it is presumed that compounds structurally similar to these reversible inhibitors, which are being tested for irreversible binding, would also bind to the ATP site on the enzyme, not the peptide-binding site. However, to rule out ATP non-competitive binding by the inhibitor, i.e., binding to the peptide site, competition assays with compounds known or predicted to bind to the ATP-binding site, such as staurosporine, can be utilized.

The Dialysis Enzyme Assay

Another assay to identify those compounds that irreversibly bind to the tyrosine kinase involves dialysis. The tyrosine kinase enzyme is incubated with the test sample and dialysed using standard techniques known in the art. A parallel sample is prepared and maintained without dialysis at the same temperature for the same amount of time. The two samples are then analyzed using the basic enzyme assay. The effect of the dialysis on the inhibition activity of the test compound is compared to the parallel non-dialysed control. If the percent inhibition activity of the test compound is the same or nearly the same for the two samples, then the test compound is likely irreversibly bound to the kinase. The principle behind this assay being that the reversibly bound test compound and enzyme can dialyze out of the bags whereas the irreversibly bound compound and enzyme camiot dialyze out of the bag. Thus, if the difference of the IC₅₀ of the dialysed and undialysed sample is about three-fold or less, the inhibitor is considered to irreversibly bind to the kinase.

The Use of Mutated Tyrosine Kinase Enzyme in the Enzyme Assay

The last assay performed to prove binding irreversibility of a potential inhibitor of the kinase also utilizes the basic enzyme assay, but rather than use a wild-type tyrosine kinase enzyme, the protein used has at least one altered, changed, deleted or added amino acid residue, or in other words, is mutated.

The terms “mutated” “mutant” and “mutation” mean any detectable change in genetic material, e.g., DNA, or any process, mechanism or result of such a change. This includes gene mutations in which the structure (e.g., DNA sequence) of a gene is altered, any gene or DNA arising from any mutation process, and any expression product (e.g., RNA, protein or enzyme) expressed by a modified gene or DNA sequence. It is understood that altered protein molecules are usually expressed in cells having one or more mutated genes that encode the altered protein.

Thus, the mutated tyrosine kinase can be produced by mutating the DNA encoding the enzyme, or by altering the RNA or protein itself. Any of these alterations or mutations can be achieved by standard recombinant DNA technology and/or protein chemistry methods.

A mutation to an amino acid residue can be made after studying the structure of the kinase and determining, through molecular modeling, the catalytic domain of the protein and the amino acid residues possibly involved in covalent binding. After this determination is made, the amino acid can be altered using standard techniques. The protein can then be cloned and transfected into cells and purified, again by standard recombinant technology techniques.

Test compounds that have appeared to bind irreversibly as shown by one or more of the assays listed above, can then be tested in the basic enzyme assay with the mutated kinase protein. It would be predicted that those compounds that inhibited the wild-type kinase and bound irreversibly would lose their activity with the enzyme mutated in the catalytic domain, where the inhibitor would covalently bind.

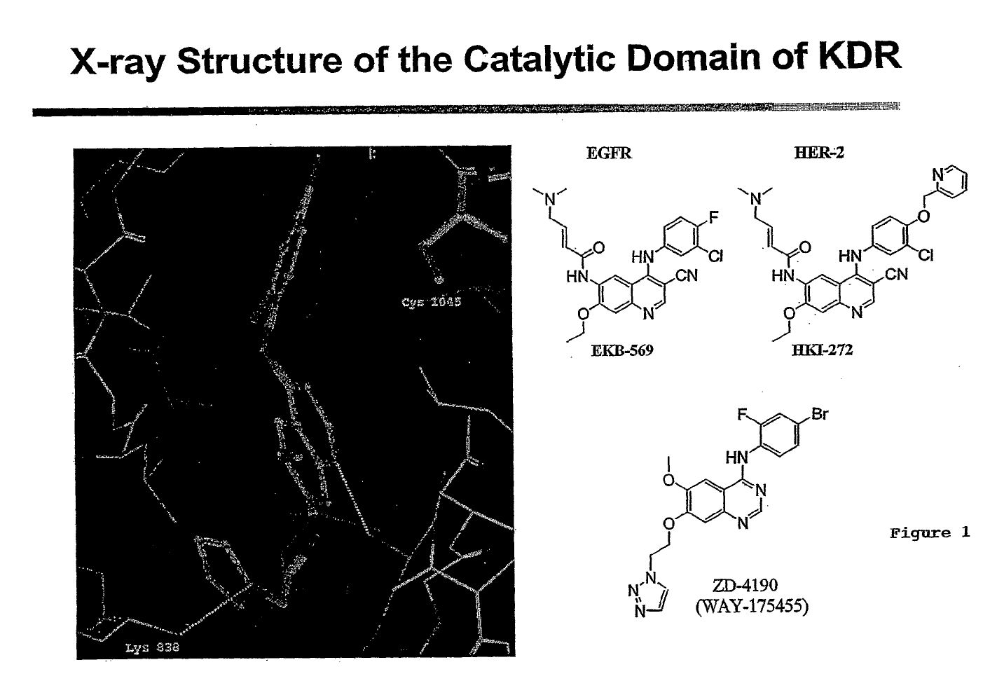

Mutants of the enzyme KDR were made based upon its crystal structure reported in McTigue et al., Structure 7:319-330 (1999). FIG. 1 shows the x-ray structure of the catalytic domain of KDR. Based upon this modeling, a cysteine at 1045 was changed to a serine or an alanine. The molecular modeling of KDR using this structure shows other amino acids, such as lysine 868, that could be mutated to study the covalent binding of potential irreversibly binding compounds. This residue can be changed from a lysine to an alanine. Furthermore, a mutated KDR with altered amino acids at both cysteine 1045 and lysine 868 could be made, especially by changing both these amino acids to alanines.

Altered tyrosine kinases can be used in the basic enzyme assay, under normal or reducing conditions, and/or in the enzyme wash-out assay, the dialysis enzyme assay and/or the ATP competition assay, using the protocols described above. The results of these assays using the altered tyrosine kinase can be compared to assays performed with the wild-type kinase.

As shown in the experimental examples, the use of the mutated KDR kinase in the enzyme assay and the wash-out assay further identified compounds which may irreversibly covalently bind to the wild type KDR.

### 6. EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing the invention in spirit or in scope. The invention is therefore to be limited only by the terms of the appended claims along with the full scope of equivalents to which those claims are entitled.

6.1 Expression of Recombinant KDR-IC-FLAG Enzyme in Human Embryonic Kidney Cells

The full cytoplasmic domain of the human KDR (VEGF-receptor 2) was cloned using standard reverse transcriptase/polymerase chain reaction (PCR) procedures. Total RNA was isolated from human umbilical vein endothelial cells (HUVEC) using RNAgents Total Isolation System (Promega). cDNA was generated using real time polymerase chain reaction (RT-PCR) (SuperScript II Rnase H-Reverse Transcriptase and Platinum Pfx DNA Polymerase, Invitrogen) and primers specific for KDR (GenBank, accession number NM_{—}002253), starting at Met-806 (underlined) (5′-ATG GAT CCA GAT GAA CTC CCA TTG) and ending at Val-1356 (underlined) (5′-AAC AGG AGG AGA GCT CAG TGT GGT). Primers were designed with HindIII/XhoI terminal sites, respectively, to allow for subcloning. The cDNA product was cloned into the pCMV-Tag4 vector (Stratagene) at the HindIII/Xhol sites, such that a FLAG sequence (AspTyrLysAspAspAspAspLys) was expressed at the C-terminus to allow for protein purification.

Human embryonic kidney (HEK) 293 cells (American Type Culture Collection) were transiently transfected with the KDR-FLAG vector and harvested 48 hours after transfection to confirm protein expression. Stable clones were then selected in geneticin G418 (500 ug/ml) for approximately three weeks and used for moderate-scale protein preparations performed as follows.

Cells (36×150 mm dishes of sub-confluent monolayers) were lysed in 72 ml of lysis buffer containing protease inhibitors (50 mM HEPES, 150 mM NaCl, 2 mM EDTA, 1% Igepal CA-630, pH 7.5, 1 mM Na₃VO₄, 1 mM PMSF, 20 KJU/ml aprotinin, 10 ug/ml pepstatin, 10 ug/ml leupeptin) and then centifuged at 12,000 rpm for 20 minutes at 4° C. to remove insoluble debris.

KDR protein was isolated from the cell lysate using batch purification on anti-FLAG M2 affinity resin (Sigma) for two hours at 4° C. followed by sequential washing and centrifugation. Resin was applied to the column and protein eluted with 200 ug/ml FLAG peptide in 50 mM HEPES, 100 mM NaCl, 10% glycerol, 1 mM Na₃VO₄, 1 mM EDTA. Fractions were collected and evaluated for KDR content by SDS-PAGE immunoblot analyses using an anti-KDR antibody as described in Dougher and Terman “Autophosphorylation of KDR in the kinase domain is required for maximal VEGF-stimulated kinase activity and receptor internalization” Oncogene 18:1619-1627 (1999) or an anti-FLAG antibody M2 antibody (Sigma).

KDR purity was typically 20-40%. Bovine serum albumin (final concentration of 1 mg/ml) and glycerol (50% v/v) were added to the purified protein and small volume aliquots were stored at −70° C.

The recombinant protein was designated KDR-IC-FLAG.

6.2 Expression of Recombinant GST-His-KDR Enzyme in Insect Cells

The full cytoplasmic domain of human KDR was cloned by standard polymerase chain reaction using first strand human placental cDNA (Invitrogen) and Advantage PCR (ClonTech). Primers were specific for KDR (GenBank, accession number NM_{—}002253) beginning at Val-805 (forward, 5′ tag cgg ccg cGT CAT GGA TCC AGA TGA ACT CCC ATT (lower case—NotI site)) and ending at Val-1356 (reverse, 5′-ttc tag aTT AAA CAG GAG GAG AGC TCA GTG TGG (lower case—aI site)). Products were subcloned into pCR2.1-Topo and transformed into E. coli cells. The plasmid DNA was isolated and the sequence verified. The NotI/KpnI sites were used for subcloning in-frame into the pAcGHLT-B vector (Pharmingen) such that a GST-His-thrombin cleavage sequence was expressed at the N-terminus to allow for protein purification.

Sf9 insect cells (Pharmingen) were transfected with the GST-His-KDR vector. The virus was collected and amplified for three cycles. Virus stock was used to infect 1-2 liter suspension cultures of Sf9 cells that were harvested 48 hours post-transfection. Cells were centrifuged and lysed using a pressure-based method in lysis buffer containing protease and phosphatase inhibitors, then centrifuged at 12,000 rpm for 20 minutes at 4° C. to remove insoluble debris.

KDR protein was purified from cell lysate by sequential column chromatography on NiNTA His-affinity resin, HiQ anion exchange, GST-affinity resin, HiQ anion exchange and finally a G3000 sizing column. Thrombin protease was used to cleave the KDR-IC domain from the N-terminal GST-His tag.

KDR purity was approximately 90% as assessed by MALDI-MS and SDS-PAGE. Final concentrations of components were: approximately 0.23 mg/ml KDR-IC protein, 25 mM HEPES, pH 7.5, 75 mM NaCl, and glycerol added to 30% (v/v). Small volume aliquots were stored at −70° C.

This recombinant cytoplasmic (intracellular) protein product was designated GST-His-KDR-IC.

6.3 KDR Kinase Enzyme Assay using the KDR-IC-FLAG Kinase

The kinase activity of the KDR-IC-FLAG was evaluated using a dissociation-enhanced lanthanide fluorescent immunoassay (DELFIA®) as described by PerkinElner Life Sciences, Boston, Mass. and in Loganzo and Hardy, “A sensitive, time-resolved fluorometric assay for detection of inhibitors of phosphotyrosine kinases” American Biotechnology Laboratory 16:26-28 (1998).

Nunc Maxisorb 96-well plates were coated at room temperature for 1 to 2 hours with 100 ul per well of 25 ug/ml poly(Glu₄-Tyr) peptide (Sigma) in tris-buffered saline (TBS) (25 mM Tris, pH 7.2, 150 mM NaCl). Unbound peptide was washed three times with TBS.

KDR-IC-FLAG enzyme was diluted from 10- to 20-fold in 0.1% BSA/4 mM HEPES. A master mix of enzyme plus kinase buffer was prepared by mixing (per well) 10 μl of diluted enzyme, 10 μl of 5× kinase buffer (20 mM HEPES, pH 7.4, 5 mM MnCl₂, 100 uM Na₃VO₄) and 9 μl of water. This master mix (29 μl) was added to each well, along with 1 μl of test compound prepared in 100% dimethyl sulfoxide (DMSO). Compounds were added as 50× stocks as necessary for single point or dose response analyses. Controls were done by adding DMSO alone, i.e., no test compound, to wells containing the master mix of enzyme plus kinase buffer.

After 15 minutes at room temperature, ATP/MgCl₂ (20 ul of 25 uM ATP, 25 mM MgCl₂, 10 mM HEPES, pH 7.4) was added to each well to initiate the reaction. Final concentrations of the assay components were: 10 uM ATP, 10 mM MgCl₂, 1 mM MnCl₂, 4 mM HEPES, pH 7.4, 20 μM Na₃VO₄, 20 ug/ml BSA, 2% DMSO.

After 40 minutes, at room temperature, the liquid was removed and the plates were washed three times with TBST (TBS with 0.05% Tween-20). The wells were then incubated for one hour at room temperature with 75 ul of 0.1 ug/ml of europium-conjugated anti-phosphotyrosine antibody (PT66, PerkinElmer) prepared in assay buffer (PerkinElmer). Plates were washed three times in TBST and then incubated for 15 minutes in the dark with 100 ul of Enhancement Solution (PerkinElmer).

Plates were read in a Victor-V multi-label counter (PerkinElmer) using the default europium detection protocol. Percent inhibition or IC₅₀ of the compounds was calculated by comparison with the DMSO-treated control wells.

6.4 KDR Kinase Enzyme Assay Using GST-His-KDR-IC Kinase

The kinase activity of the GST-His-KDR-IC kinase was also evaluated using the DELFIA® format as described in section 6.3, except 0.5 ug/ml of poly(Glu₄-Tyr) peptide substrate was used and 20 ul of 2.5 uM of ATP, to bring the final concentration of ATP in the reaction to 1 uM.

6.5 Enzyme Wash-Out Assay

To determine if the test compounds bound irreversibly to the enzyme, the plates were washed after the incubation of the enzyme and test compound and prior to the addition of the ATP.

Parallel plates were tested for each test compound wherein one plate was processed as described above in section 6.3 and the second plate was washed three times in 100 ul of 4 mM HEPES, pH 7.4, to remove unbound compound. 1× kinase buffer (30 ul 1 mM MnCl₂, 4 mM HEPES, pH 7.4, 20 μM Na₃VO₄) and 20 ul of ATP/MgCl₂ were then added to the wash-out plate. The KDR-FLAG enzyme, as described in 6.1, was used in these assays.

Detection of the phosphotyrosinylated peptide for both plates was performed as described above in section 6.3. The results are shown in Table 1. If there is little change in the IC₅₀ value in the wash-out sample (three-fold or less) compared to the sample where there is no wash-out, then it can be determined that the compound is as an irreversibly binding inhibitor. If there is a large increase in the IC₅₀ value in the wash-out experiment compared to the experiment where there is no wash-out, then it can be determined that the compound is behaving as a conventional reversible binding inhibitor.

In order to determine the behavior of conventional reversible binding KDR inhibitors in this test, the reference inhibitors Compound A and Compound B were also tested. Compound A is a quinazoline-based inhibitor reported to be a conventional ATP competitive inhibitor (Hennequin et al., J. Med. Chem., 42:5369-89 (1999) and Hennequin et al., J. Med. Chem., 45:1300-12 (2002)). Compound B is a phthalazine-based inhibitor reported to be a conventional ATP competitive inhibitor (Bold et. al., J. Med. Chem., 43:2310-23 (2000)).

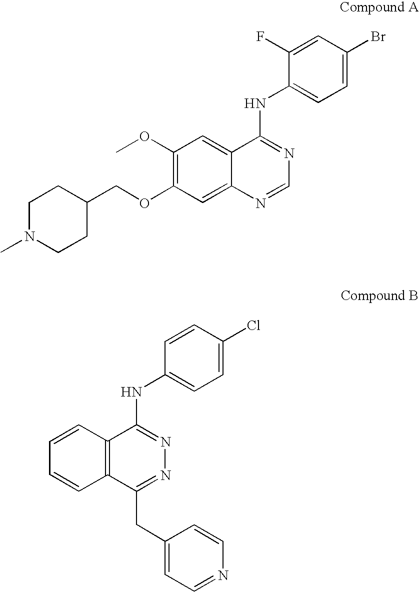

For the reference inhibitors Compound A and Compound B, it is evident from the data in Table 1 that there was a large increase in the IC₅₀ values in the experiment where there is a wash-out step compared to the experiment with no wash-out step indicating that these compounds are functioning as conventional reversible binding inhibitors. In contrast, for many of the other compounds, there was a minimal change in the IC₅₀ values between the wash-out and no wash-out experiments suggesting that these inhibitors function as irreversible binding inhibitors of the enzyme or like irreversible binding inhibitors. Some of the test compounds appeared to act like reversible binding inhibitors, but are nevertheless potent.

TABLE 1

| | IC₅₀ (nM) | |
|---|---|---|
| | NO WASH | IC₅₀ (nM) |
| COMPOUND | OUT | WASH-OUT |
| 2-[(6,7-dimethoxy-4-quinazolinyl) | 285.2 | >1000 |
| amino]-5-methylbenzo-1,4-quinone | | |
| 2-[(6,7-dimethoxy-4-quinazolinyl) | 2.3 | 1.2 |
| amino]-6-methylbenzo-1,4-quinone | | |
| 2-{[6-methoxy-7-(2-methoxyethoxy)- | 154.2 | >1000 |
| 4-quinazolinyl]amino}-5-methylbenzo- | | |
| 1,4-quinone | | |
| 2-{[6-methoxy-7-(2-methoxyethoxy) | 3.7 | 5.2 |
| quinazolin-4-yl]amino}-5-phenoxybenzo- | | |
| 1,4-quinone | | |
| 2-anilino-5-[(6,7-dimethoxy | 40.7 | 57.1 |
| quinazolin-4-yl) | | |
| amino]benzo-1,4-quinone | | |
| 2-{[6-methoxy-7-(2-methoxyethoxy) | 146.5 | 513.5 |
| quinazolin-4-yl]amino}-5- | | |
| [(4-methoxyphenyl)(methyl)amino] | | |
| benzo-1,4-quinone | | |
| 2-{[6-methoxy-7-(2-methoxyethoxy) | 95.9 | 150 |
| quinazolin-4-yl]amino}-5-[(-4- | | |
| methoxyphenyl)(methyl)amino] | | |
| benzo-1,4-quinone | | |
| 2-{[6-methoxy-7-(2-methoxyethoxy) | 8.8 | 18.5 |
| quinazolin-4-yl]amino}-5- | | |
| (2-methylphenoxy)benzo-1,4-quinone | | |
| 2-{[6-methoxy-7-(2-methoxyethoxy) | 375.1 | 693.7 |
| quinazolin-4-yl]amino}-5-piperidin-1- | | |
| yl-benzo-1,4-quinone | | |
| 2-{[6-methoxy-7-(2-methoxyethoxy) | 18.9 | 18.9 |
| quinazolin-4-yl]amino}-5-(pyridin-3- | | |
| yloxy)benzo-1,4-quinone | | |
| 2-{[6-methoxy-7-(2-methoxyethoxy) | 75.7 | 155 |
| quinazolin-4-yl]amino}-5[methyl | | |
| (phenyl)amino]benzo-1,4-quinone | | |
| 2-[[4-(dimethylamino)phenyl](methyl) | 93 | 160.9 |
| amino]-5-{[6-methoxy-7- | | |
| (2-methoxyethoxy)quinazolin-4-yl]amino} | | |
| benzo-1,4-quinone | | |
| 2-{[6,7-dimethoxyquinazolin- | 4.2 | 6.5 |
| 4-yl]amino}-5-phenoxybenzo- | | |
| 1,4-quinone | | |
| 2-[4-(1H-imidazol-1-yl)phenoxy]-5- | 12 | 27.5 |
| {6-methoxy-7-(2-methoxyethoxy) | | |
| quinazolin-4-yl]amino}benzo-1,4-quinone | | |
| 2-[4-(1H-imidazol-1-yl)phenoxy]-5- | 8.1 | 14.1 |
| {6-methoxy-7-(2-methoxyethoxy) | | |
| quinazolin-4-yl]amino}benzo-1,4-quinone | | |
| 2-[4-(1H-imidazol-1-yl)phenoxy]-5- | 2.3 | 5.3 |
| {6-methoxy-7-(2-methoxyethoxy) | | |
| quinazolin-4-yl]amino}benzo-1,4- | | |
| quinone | | |
| 5-methoxy-3-{[6-methoxy- | 17.9 | 33.1 |
| 7-(2-methoxyethoxy)quinazolin- | | |
| 4-yl]amino}-2-(1,3-thiazoylthio) | | |
| benzo-1,4-quinone | | |
| 4-({4-[4(1H-imidazol-1-yl)phenoxy]- | 53.7 | 73.7 |
| 3,6-dioxycyclohexa-1,4-dien-1-yl} | | |
| amino)-6-methoxy-7- | | |
| (2-methoxyethyoxy)quinoline- | | |
| 3-carbonitrile | | |
| Compound A | 122.8 | >1000 |
| Compound B | 438.5 | >1000 |

6.6 Enzyme Assay ATP Competition Experiments

The assay described in section 6.3 was conducted using varying concentrations of ATP to obtain final concentrations of 1, 10, 100, and 1000 uM of ATP in the reaction. The inhibitor compound used was 2-[4-(1H-imidazol-1-yl)phenoxy]-5-{6-methoxy-7-(2-methoxyethoxy)quinazolin-4-yl]amino}benzo-1,4-quinone, an irreversible binding inhibitor (see Table 1). The IC₅₀ was determined as described in section 6.3.

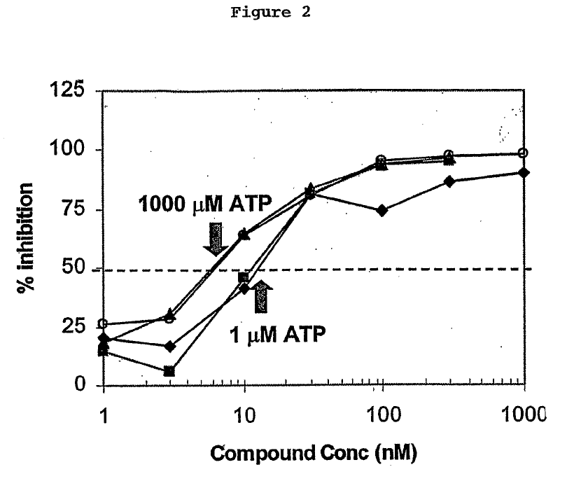

Results of this experiment are shown in FIG. 2. As shown by the graph, there was no significant change in percent inhibition of the test compound when the various concentrations of ATP were increased, suggesting that this inhibitor does not compete with ATP and binds irreversibly.

By contrast, a reversible binding inhibitor of KDR, 2-{[6-methoxy-7-(2-methoxyethoxy)-4-quinazolinyl]amino}-5-methylbenzo-1,4-quinone (see Table 1), showed a change in IC₅₀ from 169 nM in 10 uM of ATP to 840 nM in 1000 uM of ATP. These data show that this reversible binding inhibitor compound competes with ATP, which is predicted.

6.7 Enzyme Assay Dialysis Experiments

The KDR-IC-FLAG enzyme (described in section 6.1) was diluted 1:10 in BSA/HEPES and then further diluted into kinase buffer (10 ul of enzyme, 10 ul of 5× kinase buffer, 9 ul of water). Samples (145 ul of enzyme mix plus 5 ul of 25 uM test compound; final concentration of test compound in assay plate were 500 nM) were injected into a 10,000 MW cut-off dialysis chamber (Pierce) and dialyzed for 4 hours at 4° C. against 200 ml of 1× kinase buffer with three buffer changes. A parallel sample was prepared and maintained at 4° C. in a tube (no dialysis) for same time. After the incubation period, the dialysate was removed from the chamber with an 18-gauge needle and syringe. The final recovery volume was approximately 180 ul. Quadruplicates of the sample (30 ul) were added to a poly(Glu₄-Tyr)-coated plate. The non-dialyzed parallel sample was also added to the peptide-coated plate. Samples were treated and analyzed as described in section 6.3. The effect of the dialysis on compound activity against the enzyme was compared with the parallel non-dialyzed control.

The results of this assay are shown in Table 2.

TABLE 2

| | PERCENT | |
|---|---|---|
| | INHIBITION | PERCENT INHIBITION |
| COMPOUND | WITH DIALYSIS | WITHOUT DIALYSIS |
| 2-[4-(1H-imidazol-1-yl) | 56% | 86% |
| phenoxy]-5-{6-methoxy- | | |
| 7-(2-methoxyethoxy) | | |
| quinazolin-4-yl]amino} | | |
| benzo-1,4-quinone | | |
| 2-{[6-methoxy-7- | 6% | 63% |
| (2-methoxyethoxy)-4- | | |
| quinazolinyl]amino}-5- | | |
| methylbenzo-1,4-quinone | | |

These results show that 2-{4-(1H-imidazol-1-yl)phenoxy]-5-[6-methoxy-7-(2-methoxyethoxy)quinazolin-4-yl]amino}benzo-1,4-quinone, an irreversibly binding inhibitor, retains most of its activity after dialysis, suggesting that it is retained in the dialysis chamber bound to KDR. Because 2-{[6-methoxy-7-(methoxyethoxy)-4-quinazolinyl]amino}-5-methylbenzo-1,4-quinone, a known reversible inhibitor of KDR leaves the chamber, it loses most of its activity after dialysis.

6.8 Construction of KDR-Cys-1045 Mutants

Mutants of the enzyme KDR were made based upon its crystal structure reported in McTigue et al., Structure 7:319-330 (1999). FIG. 1 shows the x-ray structure of the catalytic domain of KDR. Based upon this modeling, the Cys-1045 (codon corresponding to nucleotides TGT) in the fall length KDR DNA sequence (Genbank Accession NM_{—}002253) was converted to serine (using nucleotides AGT) or to alanine (using nucleotides GCT), using the QuickChange site-directed mutagenesis kit (Stratagene). The protein was expressed in HEK293 or Sf9 cells as described for the wild type protein in sections 6.1 and 6.2. The protein was also purified using the FLAG or GST/His tags.

The protein was tested for kinase activity using the DELFIA® assay described in sections 6.3 and 6.4. The mutated protein was found to be enzymatically active in the in vitro kinase assay. This protein was designated KDR-Cys-1045.

6.9 Use of KDR-C1045A Mutant Enzyme in Enzyme and Wash-Out Assay

Test compounds were assayed using the protocol described in section 6.4 for the basic enzyme assay using the GST-His-KDR-IC enzyme and section 6.5 for the enzyme wash-out assay, except rather than the wild-type KDR enzyme, an enzyme mutated by converting the cysteine at 1045 to alanine, was used. This mutated protein was designated KDR-C1045A. Additionally, for comparison, the test compounds were assayed using the KDR wild type enzyme in both a basic enzyme assay as well as the enzyme wash-out assay. Those compounds that were found to likely bind irreversibly (based upon the enzyme wash-out (see Table 1) and dialysis experiments (see Table 2)) were re-tested with the mutant enzyme. The results are shown in Table 3.

TABLE 3

| | | KDR-WILD | | |
|---|---|---|---|---|
| | KDR-WILD | TYPE, | KDR | KDR-C1045A, |
| COMPOUND | TYPE | WASH OUT | C1045A | WASH OUT |
| 4-[(4-fluoro- | 63.2 ± 23.5 (3) | 276.8 (1) | 300.9 ± 140.3 (4) | >1000 [33%] (3) |
| 2-methyl- | | | | |
| 1H-indol-5-yl)oxy]- | | | | |
| 6-methoxy-7- | | | | |
| [(1-methylpiperidin- | | | | |
| 4-yl)methoxy] | | | | |
| quinoline-3- | | | | |
| carbonitrile | | | | |
| (non-quinone) | | | | |
| 2-{[6-methoxy-7- | 187.6 ± 100.9 (6) | >1000 [36%] (2) | >1000 [39%] (4) | >1000 [17%] (4) |
| (2-methoxyethoxy)- | | | | |
| 4-quinazolinyl] | | | | |
| amino}-5- | | | | |
| methylbenzo-1, | | | | |
| 4-quinone | | | | |
| (quinone containing) | | | | |
| 2-[4-(1H-imidazol- | 9.1 ± 3.9 (7) | 18.7 ± 7.7 (3) | 790.6 ± 225.8 (4) | 793.0 ± 289.4 (4) |
| 1-yl)phenoxy]-5- | | | | |
| {6-methoxy-7-(2- | | | | |
| methoxyethoxy) | | | | |
| quinazolin-4-yl] | | | | |
| amino}benzo-1,4- | | | | |
| quinone | | | | |
| (quinone containing) | | | | |
| 2-chloro-3-methoxy- | 0.8 ± 0.4 (3) | 1.1 (1) | 37.5 ± 14.5 (4) | 69.9 ± 28.3 (4) |
| 5-{[6-methoxy-7- | | | | |
| (2-methoxyethoxy) | | | | |
| quinazolin-4-yl]amino} | | | | |
| benzo-1,4-quinone | | | | |
| (quinone containing) | | | | |

Data are mean IC₅₀ (nM)±standard deviation for the indicated number of experiments (N). If 50% inhibition could not be achieved, the percent inhibition at the high dose tested is indicated in the brackets.

The known benchmark reversible non-quinone containing KDR inhibitor, 4-[(4-fluoro-2-methyl-1H-indol-5-yl)oxy]-6-methoxy-7-[(1-methylpiperidin-4-yl)methoxy]quinoline-3-carbonitrile, inhibited the wild type KDR with an IC₅₀ of about 63 (Table 3). However, this benchmark compound was partially washed out in the enzyme wash-out assay. Moreover, the reversible quinone-containing inhibitor, 2-{[6-methoxy-7-(2-methoxyethoxy)-4-quinazolinyl]amino}-5-methylbenzo-1,4-quinone, was also partially washed out using the wild type KDR, losing greater than five times its activity.

The irreversible quinone-containing compounds, 2-[4-1H-imidazol-1-yl)phenoxy]-5-{6-methoxy-7-(2-methoxyethoxy)quinazolin-4-yl]amino}benzo-1,4-quinone and 2-chloro-3-methoxy-5-{[6-methoxy-7-(2-methoxyethoxy)quinazolin-4-yl]amino}benzo-1,4-quinone, are highly potent against the wild-type KDR and upon wash out, retain most of their activity (only 1.4 to 2.0 times loss of activity). These data suggest that quinone-containing compounds that are predicted to bind covalently to KDR potently inhibit the enzyme, even after the unbound compound is washed away.

Compounds were then tested for activity in the basic enzyme assay and wash out assay using the KDR-C1045A mutated enzyme. The benchmark reversible non-quinone containing inhibitor, 4-[(4-fluoro-2-methyl-1H-indol-5-yl)oxy]-6-methoxy-7-[(1-methylpiperidin-4-yl)methoxy]quinoline-3-carbonitrile, retained partial activity in the basic enzyme assay using the mutant versus the wild type KDR, with less than a five times loss of activity. The reversible quinone-containing compound, 2-{[6-methoxy-7-(2-methoxyethoxy)-4-quinazolinyl]amino}-5-methylbenzo-1,4-quinone, also retained partial activity, with about a five times loss of activity.

In contrast, the irreversible quinone-containing compounds, 2-[4-1H-imidazol-1-yl)phenoxy]-5-{6-methoxy-7-(2-methoxyethoxy)quinazolin-4-yl]amino}benzo-1,4-quinone and 2-chloro-3-methoxy-5-{[6-methoxy-7-(2-methoxyethoxy)quinazolin-4-yl]amino}benzo-1,4-quinone, lost significant activity in the basic enzyme assay when the mutated KDR enzyme was used (about 87 times and 47 times loss of activity, respectively). These data suggest that the residue Cys1045 is required for potent activity of quinone-containing compounds, but is not as critical for non-quinone-containing compounds.

After the wash out assay using the mutant KDR, the quinone-containing compounds retain much of their activity against KDR-C1045A (losing either no activity or as little as 1.8 times loss of activity), suggesting that other amino acids in KDR, in addition to Cys1045, may also contribute to the binding of these compounds.

### REFERENCES CITED

Numerous references, including patents, patent applications and various publications, are cited and discussed in the description of this invention. The citation and/or discussion of such references is provided merely to clarify the description of the present invention and is not an admission that any such reference is “prior art” to the invention described here. All references cited and/or discussed in this specification (including references, e.g., to biological sequences or structures in the GenBank, PDB or other public databases) are incorporated herein by reference in their entirety and to the same extent as if each reference was individually incorporated by reference.

1. An assay for identifying a compound which inhibits the activity of and binds irreversibly to a tyrosine kinase enzyme, comprising the steps of:
a) incubating a mixture comprising the tyrosine kinase enzyme and a test compound in a substrate-coated plate well under conditions wherein, in the absence of the test compound, phosphorylation of the substrate by the tyrosine kinase enzyme would normally occur;
b) adding a wash solution to the mixture of step a) to wash out any test compound not bound to the tyrosine kinase enzyme;
c) adding ATP to the mixture of step a);
d) incubating the plate wells with an antibody to the phosphorylated substrate, wherein the antibody is coupled to a label;
e) detecting the amount of phosphorylated substrate; and
f) determining the level of phosphorylated substrate in the presence of the test compound after step b) relative to the level of phosphorylated substrate in the presence of the test compound in a sample performed without step b),
wherein a difference of about three-fold or less indicates that the test compound binds irreversibly to the tyrosine kinase enzyme.

2. The assay of claim 1, wherein the wash solution is a buffer.

3. The assay of claim 1, wherein step b) is performed more than one time

4. The assay of claim 1, wherein the tyrosine kinase enzyme is selected from a group consisting of vascular endothelial growth factor receptor-1 (VEGFR-1), vascular endothelial growth factor receptor-2 (VEGFR-2 or KDR), vascular endothelial growth factor receptor-3 (VEGFR-3), platelet derived growth factor receptor (PDGFR), fibroblast growth factor receptor (FGFR) and epidermal growth factor receptor (EGFR).

5. The assay of claim 1, wherein the tyrosine kinase enzyme is recombinant.

6. The assay of claim 1, wherein the tyrosine kinase enzyme further comprises at least one tag sequence.

7. The assay of claim 6, wherein the tag is selected from the group consisting of a-tubilin, B-tag, E-tag, c-myc, FLAG eptitope, HA, H is, HSV, PK-tag, Protein C, T7, VSV-G and GST.

8. The assay of claim 1, wherein the substrate is poly(Glu₄₋Tyr) peptide.

9. The assay of claim 1, wherein the concentration of ATP added in step c) is from about 1 nM to 10 mM.

10. The assay of claim 1, wherein the concentration of ATP added in step c) is from 0.1 uM to 100 uM.

11. The assay of claim 1, wherein the concentration of ATP added in step c) is 10 uM.

12. The assay of claim 1, wherein the label is selected from the group consisting of fluorescent labels, enzymes, fluorophores, chromophores, radioisotopes, dyes, colloidal gold, colloidal carbon, latex particles and chemiluminescent agents.

13. The assay of claim 12, wherein the fluorescent label is selected from the group consisting of terbium, dysprosium, europium and samarium.

14. The assay of claim 1, wherein the reaction of step a) occurs in a multi-well plate assay as part of a high-throughput screen.

15. The assay of claim 1, wherein the difference in the level of phosphorylated substrate in the presence of the test compound after step b) relative to the level of phosphorylated substrate in the presence of the test compound in a sample performed without step b), is two-fold or less.

16. An assay for identifying a compound which inhibits the activity of and binds irreversibly to a tyrosine kinase enzyme, comprising the steps of:
a) incubating a mixture comprising the tyrosine kinase enzyme and a test compound in a substrate-coated plate well under conditions wherein, in the absence of the test compound, phosphorylation of the substrate by the tyrosine kinase enzyme would normally occur;
b) adding ATP to the mixture of step a), in at least two increasing varying concentrations;
c) incubating the plate wells with an antibody to the phosphorylated substrate, wherein the antibody is coupled to a label;
d) detecting the amount of phosphorylated substrate; and
e) determining the level of phosphorylated substrate in the presence of the test compound and the varying increasing concentrations of ATP,
wherein a difference of about three-fold or less in the level of phosphorylation of the substrate in the varying increasing concentrations of ATP indicates that the test compound binds irreversibly to the tyrosine kinase enzyme.

17. The assay of claim 16, wherein the concentrations of ATP added in step b) are from about 1 nM to 10 mM.

18. The assay of claim 16, wherein the concentrations of ATP added in step b) are from 0.1 uM to 1000 uM.

19. The assay of claim 16, wherein the concentrations of ATP added in step b) are 1, 10, 100 and 1000 uM.

20. The assay of claim 16, wherein the tyrosine kinase enzyme is selected from a group consisting of vascular endothelial growth factor receptor-1 (VEGFR-1), vascular endothelial growth factor receptor-2 (VEGFR-2 or KDR), vascular endothelial growth factor receptor-3 (VEGFR-3), platelet derived growth factor receptor (PDGFR), fibroblast growth factor receptor (FGFR) and epidermal growth factor receptor (EGFR).

21. The assay of claim 16, wherein the tyrosine kinase enzyme is recombinant.

22. The assay of claim 16, wherein the tyrosine kinase enzyme further comprises at least one tag sequence.

23. The assay of claim 22, wherein the tag is selected from the group consisting of a-tubilin, B-tag, E-tag, c-myc, FLAG eptitope, HA, His, HSV, PK-tag, Protein C, T7, VSV-G and GST.

24. The assay of claim 16, wherein the substrate is poly(Glu₄-Tyr) peptide.

25. The assay of claim 16, wherein the label is selected from the group consisting of fluorescent labels, enzymes, fluorophores, chromophores, radioisotopes, dyes, colloidal gold, colloidal carbon, latex particles and chemiluminescent agents.

26. The assay of claim 25, wherein the fluorescent label is selected from the group consisting of terbium, dysprosium, europium and samarium.

27. The assay of claim 16, wherein the reaction of step a) occurs in a multi-well plate assay as part of a high-throughput screen.

28. An assay for identifying a compound which inhibits the activity of and binds irreversibly to a tyrosine kinase enzyme, comprising the steps of:
a) incubating a mixture comprising a tyrosine kinase enzyme and a test compound and subjecting the mixture to dialysis;
b) placing the dialyzed mixture in a substrate-coated plate well under conditions wherein, in the absence of the test compound, phosphorylation of the substrate by the tyrosine kinase enzyme would normally occur;
c) adding ATP to the reaction mixture of step a);
d) incubating the plate wells with an antibody to the phosphorylated substrate, wherein the antibody is coupled to a label;
e) detecting the amount of phosphorylated substrate; and
f) determining the level of phosphorylated substrate in the presence of the test compound in the mixture subject to dialysis relative to the level of phosphorylated substrate in the presence of the test compound not subject to dialysis, wherein a difference of about three-fold or less indicates that the test compound binds irreversibly to the tyrosine kinase enzyme.

29. The assay of claim 28 wherein the tyrosine kinase enzyme is selected from a group consisting of vascular endothelial growth factor receptor-1 (VEGFR-1), vascular endothelial growth factor receptor-2 (VEGFR-2 or KDR), vascular endothelial growth factor receptor-3 (VEGFR-3), platelet derived growth factor receptor (PDGFR), fibroblast growth factor receptor (FGFR) and epidermal growth factor receptor (EGFR).

30. The assay of claim 28, wherein the tyrosine kinase enzyme is recombinant.

31. The assay of claim 28, wherein the tyrosine kinase enzyme further comprises at least one tag sequence.

32. The assay of claim 31, wherein the tag is selected from the group consisting of a-tubilin, B-tag, E-tag, c-myc, FLAG eptitope, HA, His, HSV, PK-tag, Protein C, T7, VSV-G and GST.

33. The assay of claim 28, wherein the substrate is poly(Glu₄-Tyr) peptide.

34. The assay of claim 28, wherein the concentration of ATP added in step c) is from about 1 nM to 10 mM.

35. The assay of claim 28, wherein the concentration of ATP added in step c) is from about 0.1 uM to 100 uM.

36. The assay of claim 28, wherein the concentration of ATP added in step c) is 10 uM.

37. The assay of claim 28, wherein the label is selected from the group consisting of fluorescent labels, enzymes, fluorophores, chromophores, radioisotopes, dyes, colloidal gold, colloidal carbon, latex particles and chemiluminescent agents.

38. The assay of claim 37, wherein the fluorescent label is selected from the group consisting of terbium, dysprosium, europium and samarium.

39. The assay of claim 28, wherein the reaction of step a) occurs in a multi-well plate assay as part of a high-throughput screen.

40. An assay for identifying a compound which inhibits the activity of and binds irreversibly to a tyrosine kinase enzyme, comprising the steps of:
a) incubating a mixture comprising the tyrosine kinase enzyme that comprises at least one altered amino acid and a test compound in a substrate-coated plate well under conditions wherein, in the absence of the test compound, phosphorylation of the substrate by the tyrosine kinase enzyme would normally occur;
b) adding ATP to the reaction mixture of step a);
c) incubating the plate wells with an antibody to the phosphorylated substrate, wherein the antibody is coupled to a label;
d) detecting the amount of phosphorylated substrate; and
e) determining the level of phosphorylated substrate in the presence of the test compound and the tyrosine kinase enzyme comprising at least one altered amino acid relative to the level of phosphorylated substrate in the presence of the test compound and unaltered tyrosine kinase enzyme,
wherein a decrease in the level of phosphorylation of the substrate indicates that the test compound binds to the amino acid in the tyrosine kinase enzyme that has been altered and binds irreversibly to the unaltered tyrosine kinase enzyme.

41. The assay of claim 40, wherein the tyrosine kinase enzyme is selected from a group consisting of vascular endothelial growth factor receptor-1 (VEGFR-1), vascular endothelial growth factor receptor-2 (VEGFR-2 or KDR), vascular endothelial growth factor receptor-3 (VEGFR-3), platelet derived growth factor receptor (PDGFR), fibroblast growth factor receptor (FGFR) and epidermal growth factor receptor (EGFR).

42. The assay of claim 40, wherein the tyrosine kinase enzyme is recombinant.

43. The assay of claim 40, wherein the tyrosine kinase enzyme further comprises at least one tag sequence.

44. The assay of claim 43, wherein the tag is selected from the group consisting of a-tubilin, B-tag, E-tag, c-myc, FLAG eptitope, HA, His, HSV, PK-tag, Protein C, T7, VSV-G and GST.

45. The assay of claim 40, wherein the substrate is poly(Glu₄-Tyr) peptide.

46. The assay of claim 40, wherein the concentration of ATP added in step b) is from about 1 nM to 10 mM.

47. The assay of claim 40, wherein the concentration of ATP added in step b) is from about 0.1 uM to 100 uM.

48. The assay of claim 40, wherein the concentration of ATP added in step b) is 10 uM.

49. The assay of claim 40, wherein the label is selected from the group consisting of fluorescent labels, enzymes, fluorophores, chromophores, radioisotopes, dyes, colloidal gold, colloidal carbon, latex particles and chemiluminescent agents.

50. The assay of claim 49, wherein the fluorescent label is selected from the group consisting of terbium, dysprosium, europium and samarium.

51. The assay of claim 40, wherein the reaction of step a) occurs in a multi-well platre assay as part of a high-throughput screen.

52. The assay of claim 40, wherein the tyrosine kinase enzyme with an altered amino acid is KDR.

53. The assay of claim 52, wherein the altered amino acid residue is cysteine 1045.

54. The assay of claim 53, wherein the altered amino acid is the cysteine 1045 changed to an alanine.

55. The assay of claim 53, wherein the altered amino acid is the cysteine 1045 changed to serine.

56. The assay of claim 52, wherein the altered amino acid residue is lysine 868.

57. The assay of claim 56, wherein the altered amino acid is the lysine 868 changed to alanine.

58. The assay of claim 52, wherein the altered amino acids are lysine 868 and cysteine 1045.

59. The assay of claim 58, wherein the altered amino acids are the lysine 868 changed to an alanine and the cysteine 1045 changed to an alanine or a serine.

60. The method of claim 40, comprising the additional step of washing the mixture of altered tyrosine kinase enzyme and test compound with a wash solution, after the incubation of step a) and prior to the addition of ATP in step b).

61. A method for identifying a compound that inhibits the activity of and binds irreversibly to a tyrosine kinase enzyme, comprising performing at least two of the assays for identifying a compound which inhibits the activity of and binds irreversibly to a tyrosine kinase enzyme, wherein the assays are selected from the group consisting of:
(1) an assay comprising the steps of:
(a) incubating a mixture comprising the tyrosine kinase enzyme and a test compound in a substrate-coated plate well under conditions wherein in the absence of the test compound, phosphorylation of the substrate by the tyrosine kinase enzyme would normally occur;
(b) adding a wash solution to the mixture of step a) to wash out any test compound not bound to the tyrosine kinase enzyme;
(c) adding ATP to the mixture of step a);
incubating the plate wells with an antibody to the phosphorylated substrate, wherein the antibody is coupled to a label;
(d) detecting the amount of phosphorylated substrate; and
(e) determining the level of phosphorylated substrate in the presence of the test compound after step b) relative to the level of phosphorylated substrate in the presence of the test compound in a sample performed without step b)
wherein a difference of about three-fold or less indicates that the test compound binds irreversibly to the tyrosine kinase enzyme,
(2) an assay comprising the steps of:
(a) incubating a mixture comprising the tyrosine kinase enzyme and a test compound in a substrate-coated plate well under conditions wherein, in the absence of the test compound, phosphorylation of the substrate by the tyrosine kinase enzyme would normally occur;
(b) adding ATP to the mixture of step a), in at least two increasing varying concentrations;
incubating the plate wells with an antibody to the phosphorylated substrate, wherein the antibody is coupled to a label;
(c) detecting the amount of phosphorylated substrate; and
(d) determining the level of phosphorylated substrate in the presence of the test compound and the varying increasing concentrations of ATP,
wherein a difference of about three-fold or less in the level of phosphorylation of the substrate in the varying increasing concentrations of ATP indicates that the test compound binds irreversibly to the tyrosine kinase enzyme;
(3) an assay comprising the steps of:
(a) incubating a mixture comprising the tyrosine kinase enzyme that comprises at least one altered amino acid and a test compound in a substrate-coated plate well under conditions wherein, in the absence of the test compound, phosphorylation of the substrate by the tyrosine kinase enzyme would normally occur;
(b) adding ATP to the reaction mixture of step a):
(c) incubating the plate wells with an antibody to the phosphorylated substrate, wherein the antibody is coupled to a label;
(d) detecting the amount of phosphorylated substrate; and
(e) determining the level of phosphorylated substrate in the presence of the test compound and the tyrosine kinase enzyme comprising at least one altered amino acid relative to the level of phosphorylated substrate in the presence of the test compound and unaltered tyrosine kinase enzyme,
wherein a decrease in the level of phosphorylation of the substrate indicates that the test compound binds to the amino acid in the tyrosine kinase enzyme that has been altered and binds irreversibly to the unaltered tyrosine kinase enzyme; and
(4) an assay comprising the steps of:
(a) incubating a mixture comprising the tyrosine kinase enzyme that comprises at least one altered amino acid and a test compound in a substrate-coated plate well under conditions wherein, in the absence of the test compound, phosphorylation of the substrate by the tyrosine kinase enzyme would normally occur;
(b) adding ATP to the reaction mixture of step a);
(c) incubating the plate wells with an antibody to the phosphorylated substrate, wherein the antibody is coupled to a label:
(d) detecting the amount of phosphorylated substrate; and
(e) determining the level of phosphorylated substrate in the presence of the test compound and the tyrosine kinase enzyme comprising at least one altered amino acid relative to the level of phosphorylated substrate in the presence of the test compound and unaltered tyrosine kinase enzyme,
wherein a decrease in the level of phosphorylation of the substrate indicates that the test compound binds to the amino acid in the tyrosine kinase enzyme that has been altered and binds irreversibly to the unaltered tyrosine kinase enzyme.

62. (canceled)

63. (canceled)

64. (canceled)

65. The method of claim 1, wherein the mixture comprising the tyrosine kinase enzyme and the test compound further comprises a reducing agent.

66. The method of claim 16, wherein the mixture comprising the tyrosine kinase enzyme and the test compound further comprises a reducing agent.

67. The method of claim 28, wherein the mixture comprising the tyrosine kinase enzyme and the test compound further comprises a reducing agent.

68. The method of claim 40, wherein the mixture comprising the tyrosine kinase enzyme that comprises at least one altered amino acid and the test compound further comprises a reducing agent.

69. The method of claim 1, wherein the tyrosine kinase enzyme is a mutated tyrosine kinase enzyme.

70. The method of claim 16, wherein the tyrosine kinase enzyme is a mutated tyrosine kinase enzyme.

71. The method of claim 28, wherein the tyrosine kinase enzyme is a mutated tyrosine kinase enzyme.


SEQUENCE LISTING (6 sequences)

SEQ ID NO: 1 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 2 - protein, 1338 aa, Homo sapiens
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLELITLTCTCVAATLFWLLLTLLIRKMKRSSSEIKTDYLSIIMDPDEVPLDEQCERLPYDASKWEFARERLKLGKSLGRGAFGKVVQASAFGIKKSPTCRTVAVKMLKEGATASEYKALMTELKILTHIGHHLNVVNLLGACTKQGGPLMVIVEYCKYGNLSNYLKSKRDLFFLNKDAALHMEPKKEKMEPGLEQGKKPRLDSVTSSESFASSGFQEDKSLSDVEEEEDSDGFYKEPITMEDLISYSFQVARGMEFLSSRKCIHRDLAARNILLSENNVVKICDFGLARDIYKNPDYVRKGDTRLPLKWMAPESIFDKIYSTKSDVWSYGVLLWEIFSLGGSPYPGVQMDEDFCSRLREGMRMRAPEYSTPEIYQIMLDCWHRDPKERPRFAELVEKLGDLLQANVQQDGKDYIPINAILTGNSGFTYSTPAFSEDFFKESISAPKFNSGSSDDVRYVNAFKFMSLERIKTFEELLPNATSMFDDYQGDSSTLLASPMLKRFTWTDSKPKASLKIDLRVTSKSKESGLSDVSRPSFCHSSCGHVSEGKRRFTYDHAELERKIACCSPPPDYNSVVLYSTPPI

SEQ ID NO: 3 - protein, 1363 aa, Homo sapiens
MQRGAALCLRLWLCLGLLDGLVSDYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGPYLCSVCRPKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEQRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRVRDGTRMRAPELATPAIRHIMLNCWSGDPKARPAFSELVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 4 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 5 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 6 - protein, 1210 aa, Homo sapiens [sequence omitted]